(19) United States
(12) Plant Patent Application Publication
(10) Pub. No.: US 2014/0380533 P1
(43) Pub. Date: Dec. 25, 2014

(54) Arbutus plant named 'MBAV13'

(50) ;

(71) Applicant: Manuel Morales, Royal Oaks, CA (US)

(72) Inventor: Manuel Morales, Royal Oaks, CA (US)

(21) Appl. No.: 13/986,998

(22) Filed: Jun. 21, 2013

(51) Int. Cl. A01H 5/00

(52) U.S. Cl. PLT/216

(57) ABSTRACT

A new and distinct Arbutus cultivar named ‘MBAV13’ is disclosed, characterized by variegated leaves and small growth form. The new variety is a Arbutus, typically produced as an outdoor ornamental plant.

### LATIN NAME OF THE GENUS AND SPECIES

Arbutus

### VARIETY DENOMINATION

‘MBAV13’

### BACKGROUND OF THE INVENTION

The new cultivar is the product of chance discovery. The new variety originated as a naturally occurring branch mutation of the unpatented ‘Marina’ variety of Arbutus. The new variety was discovered by the inventor, Manuel Morales, during the Fall of 2008 in a commercial nursery in Royal Oaks, Calif. After identifying the new variety as a potentially interesting selection, the inventor continued confidential testing and observation of ‘MBAV13’ to determine the stability of the interesting foliage variegation.

Arbutus ‘Marina’ is a well-known nursery tree, introduced commercially by the Saratoga Horticultural Foundation in 1984. While the variety is well-known, the actual species has not been determined. Horticultural industry members have proposed Arbutus ‘Marina’ to either be a selection of Arbutus andracnoides or a hybrid between Arbutus. andrachnoides and Arbutus canariensis. The inventor is unaware of the actual species.

Asexual reproduction of the new cultivar ‘MBAV13’ was first performed by vegetative cuttings of the branch sport on a very limited basis beginning in late 2008. Access to all plants was restricted, as plants were kept in a location not open to the public. Through subsequent propagation, multiple generations have been reproduced, which have shown that the unique features of this cultivar are stable and reproduced true to type.

### SUMMARY OF THE INVENTION

The cultivar ‘MBAV13’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘MBAV13.’ These characteristics in combination distinguish ‘MBAV13’ as a new and distinct Arbutus cultivar:

1. Variegated foliage
2. Slow growth rate

### PARENT COMPARISON

Plants of the new cultivar ‘MBAV13’ are similar to the parent, Arbutus ‘Marina’ in most horticultural characteristics. The new variety, however, produces variegated foliage, compared to the solid colored foliage of parent ‘Marina’. Additionally, plants of ‘MBAV13’ are smaller and slower growing than plants of the parent variety

### COMMERCIAL COMPARISON

Plants of the new cultivar ‘MBAV13’ are best compared to the parent variety.

### BRIEF DESCRIPTION OF THE PHOTOGRAPHS

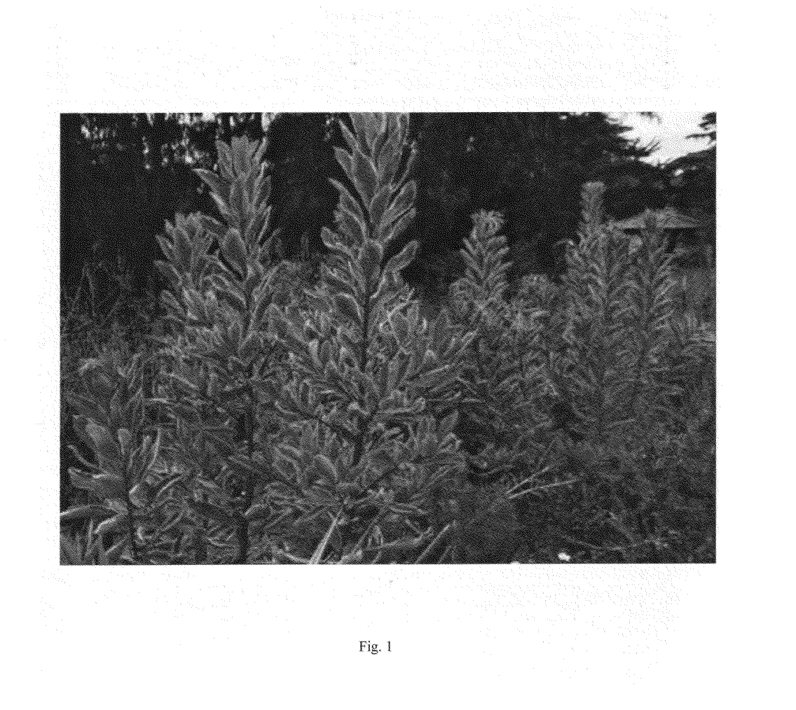

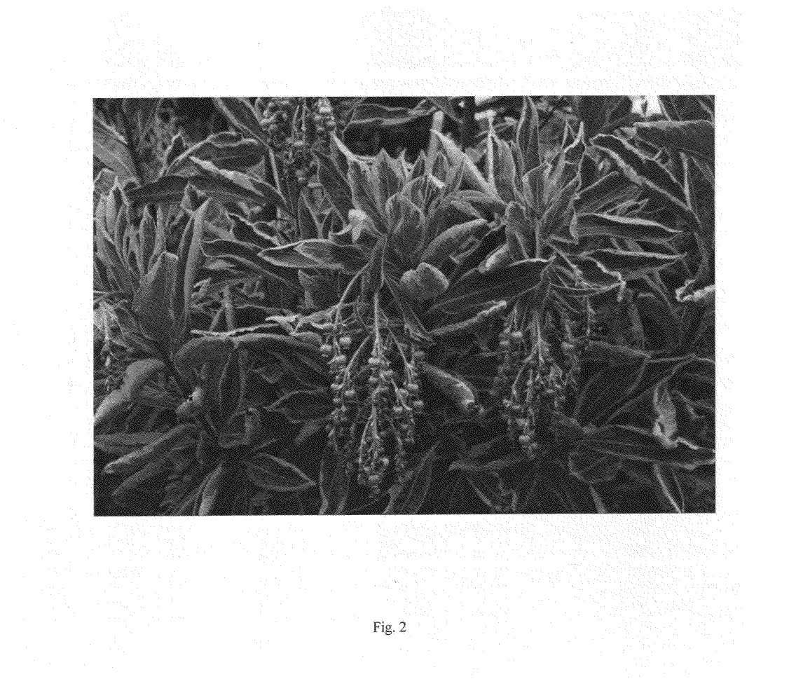

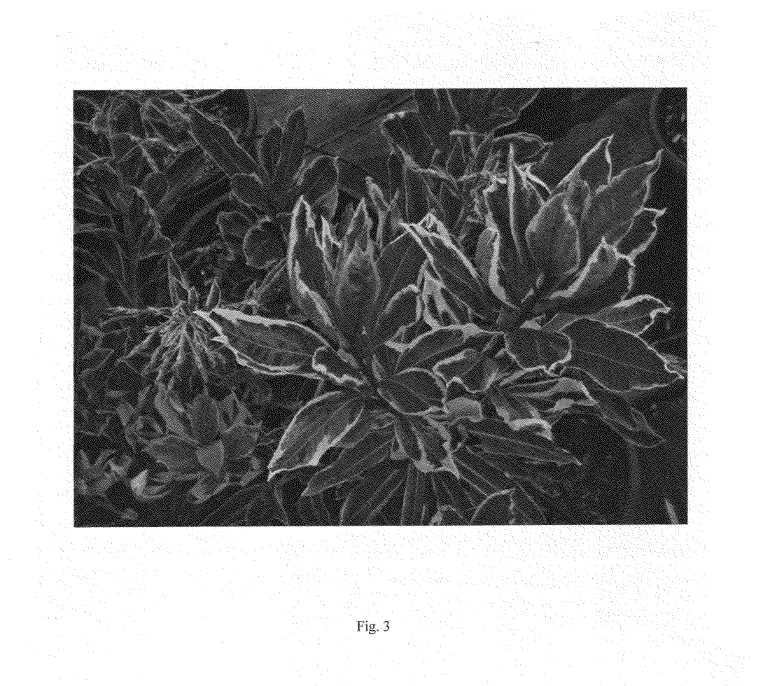

The accompanying photograph in FIG. 1 illustrates in full color a typical plants of ‘MBAV13’ grown in Royal Oaks, Calif. Plants are approximately 3 years old. FIG. 2 illustrates in full color a close up of the flowers of ‘MBAV13. FIG. 3 illustrates young plants grown in liter pots. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007, except where general terms of ordinary dictionary significance are used. The following observations and measurements describe ‘MBAV13’ plants grown outdoors in Royal Oaks, Calif. Plants are approximately 1 year old. Temperatures ranged from approximately 1° C. to 15° C. at night and 10° C. to 32° C. during the day. No artificial light, photoperiodic treatments were given to the plants. No chemical treatments were given to plants. Measurements and numerical values represent averages of typical plant types.
Botanical classification: Arbutus ‘MBAV13’

### PROPAGATION

Root description: Fine root system colored near Brown 199D and N199B

### PLANT

Growth habit: Small, often multi trunked tree.
Height: Approximately 30 cm for a one year old plant
Plant spread: Approximately 20 cm.
Growth rate: Moderate, slower growth than non-variegated forms.
Diameter of stem: Approximately 0.7 cm
Color of stem: Mature stems near RHS Brown 200B. Young growth near Greyed-Purple 187C
Texture of stem: Scaly

### FOLIAGE

Leaf:
   Arrangement.—Opposite.
   Quantity.—Approximately 14 fully expanded leaves, and 6 smaller leaves per lateral branch.
   Average leaf length.—Approximately 10 cm.
   Average leaf width.—Approximately 3.5 cm.
   Shape of blade.—Elliptic.
   Apex.—Acute.
   Base.—Cuneate.
   Margin.—Shallow serrate.
   Texture of top surface.—Glabrous.
   Texture of bottom surface.—Glabrous.
   Aspect.—Somewhat undulating.
   Color.—Young foliage upper side: Near RHS Green 143B background color, thick margin Yellow-Green 154C, fine margin Red 53B. Young foliage under side: Near RHS Green 143B background color, thick margin Yellow-Green 154C Mature foliage upper side: Near RHS Green 143B background color, thick margin Yellow-Green 154C, fine margin Red 53B. Mature foliage under side: Near RHS Green 143B background color, thick margin Yellow-Green 154C.
   Venation.—Type: Pinnate. Venation color upper side: Near RHS Yellow-Green 154C Venation color under side: Near RHS Yellow-Green 154C.
   Petiole.—Length: Average 0.8 cm. Diameter: Approximately 0.2 cm. Color: Near RHS Yellow-Green 145C Texture: Sparse, minute, stiff hairs.

### FLOWER

Natural flowering season: Spring
Length of flowering period: Approximately 8 weeks.
Inflorescence type: Mature, plants of at least 3 years old produce terminal clusters with approximately 40 individual, pendulous flowers per inflorescence. 1 year old plants produce inflorescence with approximately 10 to 20 flowers.
Individual flower type: Pendulous, urn shaped.
Bud:
   Shape.—Spherical.
   Color.—Near RHS Red 53C.
Inflorescence size, 1 year old plant:
   Diameter.—Approximately 6 cm.
   Height.—Approximately 10 cm.
Flower:
   Length.—Average 1.2 cm.
   Width.—Average 0.8 cm.
   Shape.—Bell shaped.
Flower color: Near RHS Red 54B and 54C
Pedicel:
   Length.—25 mm.
   Color.—Near RHS Red 54D.
Fragrance: None

### OTHER CHARACTERISTICS

Disease resistance: Neither resistance nor susceptibility to normal diseases and pests of Arbutus observed.
Temperature tolerance: Not tested but expected to handle a very slight frost, and high temperatures without humidity. Expected range from approximately −2° C. to 30° C.
Drought tolerance: Will tolerate some dryness once established.

What is claimed is:

1. A new and distinct cultivar of Arbutus plant named ‘MBAV13’ as herein illustrated and described.
